(19) United States
(12) Patent Application Publication
(10) Pub. No.: US 2006/0079948 A1
(43) Pub. Date: Apr. 13, 2006

(54) Hand-held ultraviolet germicidal system

(76) Inventor: Timothy Dawson, Dahlonega, GA (US)

Correspondence Address: TIM DAWSON, 564 WARD CREEK DR, DAHLONEGA, GA, 30533, US

(21) Appl. No.: 10/711,853

(22) Filed: Oct. 8, 2004

(51) Int. Cl. A61N 5/06

(52) U.S. Cl. 607/94

(57) ABSTRACT

A hand-held ultraviolet germicidal system, pen light sized. Includes an ultraviolet lamp that is enclosed in a quartz cover operated by ballast circuitry, which is powered by batteries that incorporates a timing circuit and a safety circuit. The circuitry that is powered by batteries operates a ballast which powers a UV lamp, only after the continuity of an adult hand is sensed. The unit is then held over the target area for a predetermined time for the results.

### DESCRIPTION

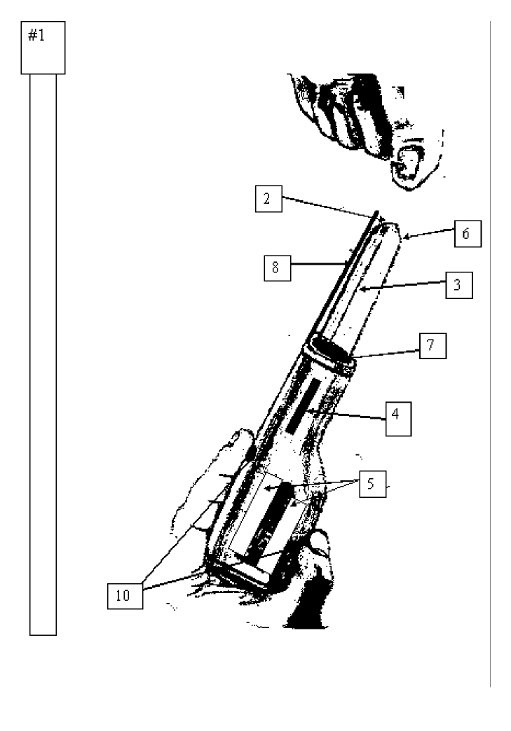

FIG. 1 illustrates a portable hand held germicidal system, pen light sized. For a method of treating onychomycosis of the human nail. #1 used to treat fungal infection of the nail, defined as the nail matrix, bed or plate.

The system includes extending from a first end #2, a pen-light size quartz UV lamp #3 that emits light in the germicidal range. In the exemplary system the lamp #3 emits light at 254 nm. The lamp is powered through ballast circuitry #4 by a battery #5 which in the example system is four AA batteries. A quartz cover #6 surrounding the UV lamp #3 fits into a holder #7 that also acts as a shock absorber for the lamp #2. The holder #7 will compresses and bend as appropriate, should the cover #2 and the lamp #3 be bumped or jarred within reason. A plastic UV filter #8 securely mounted to only one side of the pen is used to shield the user's eyes from the UV rays.

An adult hand safety sensor #10 which is incorporated in a continuity sensor circuit #4 which is connected to switches #4 between the lamp #3 and the ballast circuitry #4 and the batteries #5 prevents the UV lamp from turning on until the unit is firmly griped by an adults hand.

The plastic UV filter #8 protects the user who is holding the device #1 from harmful levels of the UV radiation, which might otherwise adversely affect his or her eyes.

The sensor #10 may, for example, be a capacitive-type sensor that senses the difference in capacitance of the moist adult hand and the surrounding air. When the sensor determines that it is firmly griped, which necessarily means that the unit #1 is firmly griped by an adults hand, the sensor closes the switches and allows the lamp to be turned on.

What is claimed is:

1. A method of treating onychomycosis by disinfecting human nails. This method includes the steps of:
Directing the UV light source and associated UV transmissive cover that extends from the first end of the system at the area of the body to be disinfected;
Sensing that the unit is held by an adult hand;
Turning the light source on to emit UV radiation in the 254 nm range directed at the area to be disinfected.

2. The method of claim i further includes the step of turning the light source off at a predetermined time after the light source is turned on.

3. A hand-held UV germicidal system comprising:
A UV light source;
A UV transmissive cover that fits over the light source;
A reflective cover to direct the UV light;
Power means for supplying power to the light source; and
A case that contains the power means and connects to the UV transmissive protective cover.

4. The system of claim iii further including a safety sensor that prevents the unit from being turned on until an adult hand properly holds it.

5. The system of claim iv further including a timing circuit that turns the light source off a predetermined time after the sensor turns the light source on.

6. The system of claim iii wherein the power means includes a battery power supply and an associated ballast circuitry.
